(19) United States
(12) Patent Application Publication
(10) Pub. No.: US 2004/0248225 A1
(43) Pub. Date: Dec. 9, 2004

(54) Luciferin hydrazides

(76) Inventors: Dieter Heindl, Paehl (DE); Rupert Herrmann, Weilheim (DE); Hans-Peter Josel, Weilheim (DE); Erasmus Huber, Finning (DE); Ursula Klause, Peissenberg (DE); Oliver Simon, Wien (AT)

Correspondence Address: Roche Diagnostics Corporation, 9115 Hague Road, PO Box 50457, Indianapolis, IN, 46250-0457, US

(21) Appl. No.: 10/479,443

(22) PCT Filed: May 28, 2002
(86) PCT No.: PCT/EP02/05854

(30) Foreign Application Priority Data
Jun. 1, 2001 (EP) 01112879.0
Feb. 15, 2002 (EP) 02003166.2

(51) Int. Cl. G01N 33/53; C12Q 1/66; C07D 237/30

(52) U.S. Cl. 435/8; 530/409; 435/7.1; 544/237

(57) ABSTRACT

The present invention relates to substituted hydrazides of a luciferin dye or to substituted hydrazides of a dye analogous thereto. These chemical compounds comprise the luciferin dye or the dye analogue thereto as a light emitting moiety precursor and the substituted hydrazide as a leaving group precursor, wherein a nitrogen atom of said hydrazide group is bound to a carbonyl group or to a group chemically equivalent to said carbonyl group of said luciferin or said analogue. The invention also relates to conjugates between a biomolecule and these compounds and to the use of such compounds in chemiluminescence procedures.

The present invention relates to substituted hydrazides of a luciferin dye or to substituted hydrazides of a dye analogous thereto. These chemical compounds comprise the luciferin dye or the dye analogue thereto as a light emitting moiety precursor and the substituted hydrazide as a leaving group precursor, wherein a nitrogen atom of said hydrazide group is bound to a carbonyl group or to a group chemically equivalent to said carbonyl group of said luciferin or said analogue. The invention also relates to conjugates between a biomolecule and these compounds and to the use of such compounds in chemiluminescence procedures.

The specific detection and quantitation of biological molecules has been accomplished with excellent sensitivity for example by the use of radio-labeled reporter molecules. The first radio immunoassays developed in the end of the 1950's have matured into the most important tools of in vitro diagnostic, especially in medicine, using a broad variety of different detection or reporter systems. Well-known examples of reporter molecules are enzymes, labeled latex beads, fluorescent dyes and especially chemiluminescent dyes.

Reviews describing the theory and practice of specific binding assays are available. The skilled artisan will find all necessary technical details for performing specific binding assays in textbooks like Tijssen, in “Practice and theory of enzyme immunoassays” (1990), Elsevier, Amsterdam and various editions of Tijssen, in “Methods in Enzymology”, Eds. S. P. Colowick, N. O. Caplan and S. P., Academic Press, dealing with immunological detection methods, especially volumes 70, 73, 74, 84, 92 and 121.

Paralleled by the development of light measuring techniques and the commercial availability of highly sensitive apparatus, luminophores have in many applications replaced isotopic labels. Some of the new luminescent labels facilitate analyte detection at extremely low levels of sensitivity. Therefore such labels also commercially are very interesting.

Luminescent labels may be subdivided into the group of fluorescent labels and the group of luminescent labels. Whereas fluorescent labels require irradiation of a sample with excitation light in order to detect and measure the fluorescent label present, the chemiluminescent systems do not require an extra source of light.

Well known chemiluminescent based systems make use of labels comprising amongst others the following categories, the combination of luciferins with corresponding luciferases, cyclic arylhydrazides, acridinium derivatives, stable dioxetanes, and oxalic acid derivatives.

Rapaport E., et al., J. Amer. Chem. Soc. 94 (1972), 3153-3159 describe the haydrazide of dehydroluciferin. They demonstrate that the hydrazide of dehydroluciferin may be substituted by an alkyl residue and report that some of the allyl derivatives are not chemiluminescent.

McCapra, et al., Chem. Comm. (1967), 22-23, investigated the chemiluminescence of dimethyl-luciferin.

White, et al., Photochem Photobiol. 53 (1991) 125-130, describe that luciferin derivatives may be conjugated and used in the detection of biochemical substances.

A preferred class of chemical compounds used in chemiluminescent labeling are luciferins or analogues thereto in combination with the corresponding luciferases (Mayer, A. and Neuenhofer, S., Luminescent labels —more than just an alternative to radioisotopes? in “Angewandte Chemie: International Edition in English” (1994) 1044-1072, Eds. E. P. Goelitz, VCH Verlagsgesellschaft mbH, Weinheim).

Several mechanisms leading to emission of light according to the chemiluminescence principles have been proposed. Short-lived intermediates are considered part of the processes leading to decarboxylation and emission of light.

One of the best known and most studied light systems in nature “operates” in the North American firefly (Photinus Pyralus). Although the mechanism of bioluminescence has been studied for more than 30 years, and the benzothiazole derivative of luciferin became available synthetically and was structurally determined at the beginning of the 1960s, not all the details of the bioluminescence reaction have been elucidated, yet.

It had been assumed for a long time and to a large extent proven at the end of the 1970s, that the specific luciferase of the firefly catalyzes the oxidation of luciferin in the presence of ATP and magnesium ions. The processes postulated for fire-fly luciferin are schematically shown in FIG. 1. Initially, a complex is formed from the acyl-AMP species of luciferin and luciferase. In the presence of oxygen oxidation ensues to given excited oxyluciferin which returns to the ground state by emitting a photon. In vivo the yellow-green emission (λₘₐₓ=565 nm) of the dianion was observed and in vivo an additional red emission (λₘₐₓ=615 nm) of the monoanion which is pH-dependent. The oxidation proceeds presumably via a dioxetanone intermediate which decarboxylates to furnish excited oxyluciferin.

To what extent one can view the often proposed dioxetanone as an intermediate or rather as a transition state is still unclear. But, all in all, the light reaction of the firefly appears to be elucidated. The assumption, first made at the end of the 1950s, that Coenzyme A also plays a role in the light reaction has been confirmed in the past few years. Addition of the coenzyme may further improve the applicability of the conventional firefly luciferin/luciferase system in the near future, since the intensity and duration of the light emission can be increased. The limiting factor for this method until now, in addition to the limited hydrolytic stability of luciferin and the sensitivity of luciferase, was the poor availability of the enzyme luciferase extracted from fireflies.

The advantageous use of a luciferin or a luciferin-derivative as indicated above, largely depends both on the stability/lability of the dye compound as well as on the availability and stability of the auxiliary enzyme(s) (e.g., luciferase, esterase, peptidase, galactosidase, aso.) used in the detection procedure.

Since luciferins and analogues thereto represent chemiluminescence dyes with very attractive basic features, it was the task of the present invention to find and identify novel compounds comprising a dye of the luciferin class or a dye analogue thereto, for use in chemiluminescence assays which provide for advantages as compared to the systems known in the art. Such advantages for example may independently be a stable dye or label, a sensitive detection, a high quantum yield and/or a chemiluminescense detection procedure not requiring any luciferase. For many applications compounds additionally comprising a coupling group are needed which are suitable for labeling of, or conjugation to a biomolecule.

Surprisingly, it has been found that compounds can be synthesized comprising a luciferin-type dye as a light emitting moiety precursor and a substituted hydrazide as a precursor of a leaving group which help to overcome problems known in the art. These novel compounds are characterized by a carbonyl-hydrazide bond between a nitrogen atom of the substituted hydrazide group and a carbonyl group or a group chemically equivalent thereto of a luciferin-type dye. Quite different to the procedures known in the art, in the present invention the procedure leading to luminescence of luciferin does not require the enzyme luciferase, and is merely based on chemical or enzymatic oxidation. In the enzymatic oxidation preferably one of the robust and well-known peroxidases, e.g., horseradish peroxidase, is used.

The compounds of the present invention are stable under routine conditions. By oxidation of the hydrazide bond of these compounds the leaving group precursor becomes activated, upon further oxidation the leaving group leaves the compound and the light emitting group precursor, the luciferin-type dye, releases energy in form of chemiluminescence.

In short the present invention relates to a chemical compound comprising a dye of the luciferin class or an analogue thereto and a hydrazide group or a substituted hydrazide group, wherein one nitrogen atom of said hydrazide group is bound to a carbonyl group or to a group chemically equivalent to said carbonyl group of said luciferin or said analogue.

Since the compounds according to the present invention encompass both storage stability, as well as sensitive detection in chemiluminescent procedures they are also used to label biomolecules and the resulting conjugates with great advantage can be applied in appropriate specific binding assays for detection of an analyte in a sample.

With great advantage the novel compounds can be used in the detection of peroxide as well as in the detection of peroxidase.

The invention also relates to a method of performing a chemiluminescence measurement using a novel compound as described in which method the leaving group precursor first is oxidized, thereupon transformed into a leaving group, the light emitting group precursor upon further oxidation becomes reactive, energy in form of light is generated and the emitted light is measured according to standard procedures. This chemiluminescence procedure based on a luciferin-type dye does not require any luciferase activity. It completely is based on chemical oxidation or on enzymatic activation using a peroxidase.

### DETAILED DESCRIPTION OF THE INVENTION

The present invention relates to a chemical compound comprising a dye of the luciferin class or an analogue thereto and a hydrazide group or a substituted hydrazide group, wherein one nitrogen atom of said hydrazide group is bound to a carbonyl group or to a group chemically equivalent to said carbonyl group of said luciferin or said analogue.

The chemical compounds according to the present invention comprise a dye of the luciferin class or a dye analogue thereto as a light emitting moiety precursor and a substituted hydrazide as a precursor of a leaving group. These two chemical entities are linked together via an amide-like bond between the carbonyl group of said dye and a nitrogen atom of said substituted hydrazide. This bond is termed “carbonyl-hydrazide bond”. This carbonyl-hydrazide bond is stable, thus ensuring the stability of the overall chemical structure, e.g., it is not hydrolyzed under physiological conditions or under routine storage conditions.

The novel dye derivatives comprising a luciferin-like dye as a light emitting moiety precursor can be easily handled, e.g., during conjugation to biomolecules or under long-term storage conditions, e.g. as required for many commercial applications.

A “light emitting moiety precursor” in the sense of the present invention comprises such chemical moieties, which upon appropriate activation can be used and measured in an analysis system based on the detection of chemiluminescence. The luciferin-type dye comprised in a compound according to the present invention is converted into a light emitting moiety upon oxidation processes as exemplified in FIG. 2.

The light emitting moiety precursor of the present invention must carry a carbonyl group or a chemically equivalent group. In a compound according to the present invention, the luciferin-like light emitting moiety precursor, of course, is not present as a free light emitting group precursor but rather it is bound to a substituted hydrazide representing the leaving group precursor. With other words the light emitting moiety precursor in the compounds described has to be understood as the luciferin carboxylic acid part of the carbonyl hydrazide bond.

The characteristic and important function of the carbonyl group is that nucleophiles, like H₂O₂, can attack the sp² carbon atom. It is well-known that groups like thiocarbonyls or cyanimino residues bring about similar chemical properties as the carbonyl group. Thiocarbonyls and cyanimino groups are groups which are considered to be “chemically equivalent to a carbonyl group”. Amongst these groups the carbonyl group and thiocarbonyl group are preferred, the carbonyl group being most preferred. In order to avoid linguistic redundancies, in the following in most cases simply the term carbonyl group is used. It has to be understood, however, that appropriate functional equivalents may as well be used.

By oxidation the stable carbonyl-hydrazide bond is converted into a labile carbonyl-N═N bond and the leaving group precursor thus is converted into the leaving group. As the term indicates, the leaving group “leaves”—after reaction of the carbonyl group with peroxide and hydrolytic cleavage —leaving back an activated luciferin (cf. FIG. 2).

The carbonyl group which is part of the stable carbonyl hydrazide bond is the same carbonyl function which (after the leaving group has been formed) upon attack by peroxide and accompanied by emission of light is cleaved off from luciferin (cf. FIG. 2).

According to the proposed mechanism the carbonyl group (which has been part of the carbonyl hydrazide bond) by attack of H₂O₂ becomes part of a dioxetanone moiety. Spontaneous decomposition of the dioxetanone moiety is accompanied by light emission and in case of a carbonyl group yields a heterocyclic ketone and CO₂, or in more general chemical terms a heterocumulene in case functional equivalents of the carbonyl group had been present.

The term “luciferin-like” dye is used to indicate two different aspects, i.e. that such dyes may either be derived from a class of dyes summarized as luciferins (see Formula 1) and from analogues thereto which are structurally quite different, but can be used in chemiluminescence procedures in an analogous manner (see Formula 2 and 3).

The term “precursor of a leaving group” is used to indicate that without further chemical modification, according to the present invention oxidation, the leaving group precursor will not function as leaving group or at least is rather a poor leaving group and essentially no light emitting moiety precursor will be set free without oxidation. Without oxidation of the hydrazide bond of the leaving group precursor the carbonyl hydrazide bond between the light emitting moiety precursor and the leaving group precursor is stable towards hydrolysis.

In the compounds according to the present invention precursors of a leaving group are used instead of a leaving group. In a preferred embodiment the compounds comprise the hydrazide group. In a further preferred embodiment the compounds comprise a substituted hydrazide group. These leaving group precursors are characterized in that they contain an “oxidizable” nitrogen as part of a carbonyl hydrazide bond.

“Oxidizable” means that said nitrogen in said carbonyl-hydrazide bond is electron rich and that electrons can be readily withdrawn, i.e. that nitrogen or that hydrazide bond thus is oxidized. As the skilled artisan will appreciate, an electron rich nitrogen requires the attachment of at least one so-called (electron) donor substituent. Electron donor substituents are well-known to the skilled artisan and need not to be detailed here. The donor substituent can be attached directly or alternatively vinylogous or phenylogous to one nitrogen atom of such a hydrazide group. In both cases the nitrogen atom is part of the reduced form of a two step donor-pi-donor redox system, also known as reversible two step redox system, as described by Huenig (Huenig, S., Pure & Appl. Chem. 62 (1990) 396-406).

In a preferred embodiment the dye of the luciferin class is a dye of Formula 1. Analogues thereto are selected from dyes represented by Formula 2 and 3.

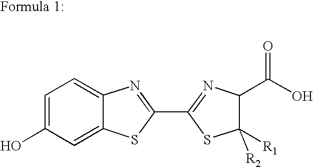

R₁=R1=H, lower alkyl(C₁-C₆),

R₂=R2=H, CH3.

In a preferred embodiment both R1 and R2 are methyl groups. Surprisingly it has been found that, e.g., the di-methyl-luciferin exhibits a flash kinetics of chemiluminescence, whereas the fire-fly luciferin (R1 and R2 are hydrogen) exhibits a long-lasting “glow-type” kinetics.

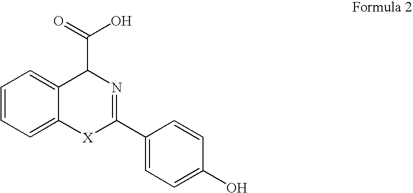

X=O 2-(4 hydroxyphenyl)4H benzo[e][1,3]oxazine-4-carboxylic acid

X=S 2-(4 hydroxyphenyl)4H benzo[e][1,3]thiazine-4-carboxylic acid

X=N-alkyl N-alkyl-2-(4 hydroxyphenyl)1,4 dihydro quinazolin 4-carboxylic acid

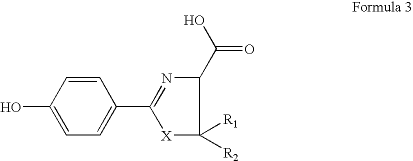

X=O 2-(4 hydroxyphenyl)4,5 dihydro oxazole 5,5 dimethyl-4-carboxylic acid

X=S 2-(4 hydroxyphenyl)4,5 dihydro thiazole 5,5 dimethyl-4-carboxylic acid

X=N-alkyl N-alkyl-2-(4 hydroxyphenyl)4,5 dihydro imidazol 5,5 dimethyl-4-carboxylic acid;

R₁ and R₂ are as defined above.

In a preferred embodiment the present invention relates to a chemical compound comprising a luciferin dye according to Formula 1 as a light emitting moiety precursor and hydrazide or a substituted hydrazide as a precursor of a leaving group, wherein the carbonyl group or a chemically equivalent group of said luciferin is linked to a hydrazide nitrogen atom of the leaving group precursor. Formula 4 gives an example of such a compound which is based on a dye of the luciferin class of dyes. Of course, the dyes analogous to luciferin as given in Formula 2 and 3 can also be used and it has to be born in mind that luciferin (or an analogue thereto) may be used in its d-, or its l-, or in racemic form.

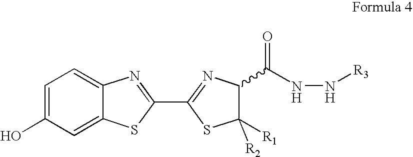

R₁=H, lower alkyl(C₁-C₆),

R₂=H, CH₃,

R₃=R₁, alkyl, alkyl-Y, aryl, aryl-Y, alkyl-aryl, alkyl-aryl-Y, heteroaryl, heteroaryl-Y, or a substituted form thereof, wherein Y=a coupling group or a label.

Preferably alkyl is a lower alkyl(C₁-C₆) and also preferred the aryl is a C₆, C₁₀ or C₁₄ aryl. Most preferred the aryl group is a phenyl group.

In case Y is a coupling group, the group Y is capable of being conjugated to a second molecule, especially a protein, a polysaccharide a polynucleotide or another biological material (see below).

In case Y is a label, preferred label molecules have a molecular weight of less than 2000 Dalton, and the labels biotin and digoxigenin are most preferred.

It is preferred that R₃ represents a residue which is electron-rich, thus facilitating the oxidation of the hydrazide bond. Such preferred groups for R₃ are residues which are embody a pi electron system as described above.

Most preferred, R₃ is an phenyl residue and carries the substituents as defined in Formula 5.

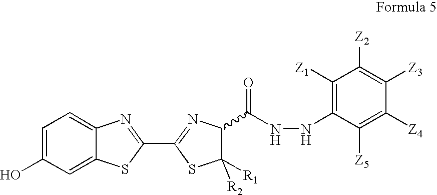

wherein R1 and R2 are as defined above,

Z1, Z2, Z3, Z4 and Z5 independently are H, Y, alkyl, alkyl-Y, aryl, aryl-Y, alky-aryl, alkyl-aryl-Y, hetreoaryl, heteroaryl-Y, OH, NH₂, O-alkyl, NH-alkyl, N(alkyl)₂, O-aryl, halogen, and/or comprising two or more of the groups Z1-Z5 as part of a carbocyclic or heterocyclic ring system, wherein Y is as defined above and is only present once in case Y is a coupling group.

Especially preferred Z1 to Z5 independently are selected from H, OH, NH₂, alkyl(C1-C6), O-alky(C1-C6), NH-alkyl(C1-C6), N(alkyl(C1-C6))₂, alkyl(C1-C6)—Y, O-alkyl(C1-C6)—Y, NH-alkyl(C1-C6)—Y, N-alkyl(C1-C6)— alkyl(C1-C6)—Y.

The compounds according to the present invention comprising a luciferin-like dye as a light emitting moiety precursor and hydrazide or a substituted hydrazide as a precursor of a leaving group linked together by carbonyl-hydrazide bond represent very attractive labels, e.g., for labeling of biomolecules. The methods used for coupling of labels to biomolecules have significantly matured during the past years and an excellent overview is given in Aslam, M. and Dent, A., The preparation of protein-protein conjugates in “Bioconjugation” (1998) 216-363, Eds. M. Aslam and A. Dent, McMillan, London and in the chapter “Macromolecule conjugation” in “Practice and theory of enzyme immunoassays” Tijssen supra. The skilled artisan knows how to make conjugates and/or will find all information necessary to make such conjugates in these textbooks.

Appropriate coupling chemistries are known from the above cited literature (Aslam, supra). The chemical compound according to the present invention preferably is designed and synthesized to comprise a coupling group which matches the coupling chemistry appropriate for the biomolecule under investigation.

In a preferred embodiment the group Y of the chemical compound according to the present invention is a coupling group. This coupling group is a reactive group or activated group which is used for chemically coupling of the compound to a biomolecule. The group Y preferably is an activated carboxylic acid group such as a carboxylic acid halogenide, a carboxylic acid anhydride, a carboxylic acid hydrazide, a carboxylic acid azide or an active ester e.g. an N-hydroxy-succinimide, a p-nitrophenyl, pentafluorophenyl, imidazolyl or N-hydroxybenzotriazolyl ester, an amine, a maleimide, a thiol, a para-aminobenzoyl group or a photoactivatable group e.g. an azide. Y is selected to match the chemical function on the biomolecule to which coupling shall be performed.

Amino groups of biomolecules (the terminal —NH₂ group or the NH₂ group of a lysine side chain, as well as ω-amino groups of diamino carboxylic acids) can be used for chemical coupling of a marker group thereto based on “amino chemistry”. Well-known examples of amino chemistry comprise amongst others the reaction of amino groups with so-called activated groups, like NHS-esters, other activated esters, acid chlorides and azides.

Carboxyl groups on biomolecules (the terminal C00⁻group, the carboxy functions of glutamic acid or aspartic acid) are used for chemical coupling based on “carboxy chemistry”. Well-known examples of carboxy chemistry comprise amongst others the activation of these of carboxy groups to carry the above mentioned activated groups. Coupling to e.g., amino groups on the marker is then easily performed.

Alternatively sulfhydryl groups on biomolecules (e.g. free-SH-groups of cysteine or —SH groups obtained by reducing di-sulfhydryl bridges) are used for chemical coupling based on “sulfhydryl chemistry”. Well-known examples of sulfhydryl chemistry comprise amongst others the reaction of —SH groups with maleimido groups, or alkylation with α-halogen carboxylic group or by thioethers.

The hydroxyl group of tyrosine residues or the imidazol group of histidine also may be used to covalent link compounds according to the present invention to a biomolecule by aid, e.g., of diazonium groups.

The coupling group may be either part of the light emitting group precursor or of the leaving group precursor. It is generally accepted that large biomolecules may interfere with the luminescence light emitted by the chemiluminescent group if both the chemiluminescent group and the biomolecule are in dose proximity. It is therefore preferred that the coupling group is part of the leaving group precursor and preferably such compounds are used for coupling to a biomolecule. In this case upon oxidation of the precursor of the leaving group the light emitting moiety precursor is released from the biomolecule and both molecules no longer are in close proximity. This is advantageous in an assay for detection of an analyte in a sample.

In general, compounds according to the invention are synthesized by reacting an activated form of the light emitting precursor, preferably an acid chloride, with the leaving group precursor in its reduced form. Chemical substances comprising hydrazides which are suitable as leaving group precursors, are commercially available or can be synthesized according to standard procedures. Preferred substituted hydrazides are substituted aryl hydrazides and most preferred substituted phenyl hydrazides.

The term “biomolecule” comprises molecules and substances of interest in a therapeutic or a diagnostic field. Biomolecule in the sense of the present invention may be any naturally occurring or synthetically produced molecule composed of biological molecules like amino acids, nucleotides, nucleosides, lipids, and/or sugars. Non-naturally occurring derivatives thereof like artificial amino acids or artificial nucleotides or nucleic acids analogs may also be used to substitute for the biomolecule.

In a preferred embodiment the biomolecule is selected from the group consisting of polypeptides, nucleic acids, and low molecular weight drugs.

A conjugate between a biomolecule and a chemical compound comprising a light emitting moiety precursor and a precursor of a leaving group with the characteristics according to the present invention, represents a further preferred embodiment. It will be readily appreciated by the skilled artisan that conjugates between a biomolecule and the chemical compounds described in the present invention is of great advantage in a specific binding assay for detection of an analyte in a sample.

Specific binding assays in general are based on the specific interaction of two members of a bioaffine binding pair. Examples of suitable binding partners in such binding pairs are hapten or antigen and an antibody reactive thereto, biotin or biotin-analogs such as amino, biotin, iminobiotin, or desthiobiotin which binds to biotin or streptavidin, sugar and lectin nucleic acid or nucleic acid analogs and complementary nucleic acid, receptor and ligand for example steroid hormone receptor and steroid hormone, and enzymes and their substrates.

The specific interaction between nucleic acids (or nucleic acid analogs) and nucleic acids complementary thereto in assays based on detection of hybridization between nucleic acid strands and the specific interaction of antibodies with their respective antigen on which the broad range of immunoassays is based, represent the most preferred binding pairs.

The theory and practice of nucleic acids hybridization assays is summarized in relevant text books, like Kessler, C., in “Non-radioactive labeling and detection of biomolecules” (1992), Springer Verlag, Heidelberg. The skilled artisan will find all relevant details therein.

Immunoassays nowadays are broadly used and general knowledge to the skilled artisan. Relevant methods and procedures are summarized in related text books, like “Bioconjugation” Aslam, M. and Dent, A. (1998) 216-363, London, McMillan Reference and “Practice and theory of enzyme immunoassays” Tijssen (1990), Amsterdam, Elsevier. A comprehensive review can also be found in an article authored by Mayer, A. and Neuenhofer, S. “Angewandte Chem. Intern. Ed. Engl.” (1994) 1063-1068, Weinheim, VCH Verlagsgesellschaft mbH.

The chemical compounds as described herein have the striking feature that the carbonyl-hydrazide bond between a light emitting moiety precursor and a precursor of a leaving group becomes unstable upon oxidation of the leaving group precursor. Light generation i.e. chemiluminescence thus is dependent on the presence of oxidants and peroxide. It therefore is evident that the chemical compounds described can be used both in assays for detection of peroxide on the one hand as well as in assays for detection of peroxidase on the other hand.

In a preferred embodiment the compounds according to the present invention are used in a method for detection of peroxide.

Peroxidase may be used to oxidize the leaving group precursor which after oxidation functions as leaving group. Under appropriate assay conditions the presence of peroxidase thus can be detected upon measurement of chemiluminescent light emitted. In a preferred embodiment the chemical compounds according to the present invention are used in a detection method based on the activity of peroxidase. Most preferred the novel compounds are used for detection of peroxidase.

Various mechanisms are at hand to oxidize the hydrazide group of the leaving group precursor. Dependent on the oxidizability of the leaving group precursor on the one hand and of the mode of application on the other hand appropriate oxidants are selected.

In a preferred mode for performing a method according to the present invention the oxidation is performed using a peroxidase.

It is also preferred to use appropriate chemical oxidants. For a measurement process according of the present invention, conditions for chemical oxidation have to be chosen which ensure that no destruction of the light emitting molecule occurs (that e.g., no break of a C—C bond takes place). Typical chemical oxidants include per-borate, per-sulfate, DDQ (dicyano dichloro quinone), diluted HNO₃, BrO₄—, H₂O₂, or cerammonium IV nitrate. As mentioned, oxidation conditions in this step must be chosen such that no destruction of the light emitting molecule occurs. Such conditions are easily established by routine experimentation.

Preferably the reagent used for oxidation of the light emitting group precursor is the same as the one used to transform the leaving group precursor to the leaving group. Most preferred oxidation is performed and light is generated by use of H₂O₂ in presence of peroxidase.

In a further preferred mode, oxidation is performed by electrochemical means.

In a further preferred embodiment the present invention relates to a method of performing a luminescence measurement based on the use of a compound according to the present invention. The method is characterized in that in the presence of peroxide the leaving group precursor is oxidized, the light emitting group precursor is activated, energy is emitted and measured.

The chemical compounds according to the present invention do not comprise an active leaving group. The leaving group precursor has to be oxidized and its oxidized form works as a leaving group. This refers to the oxidative step transforming the leaving group precursor into the leaving group. In case of donor-pi-donor leaving groups this means that redox processes according to the Wurster or Weitz type occur (Huenig, supra).

The light emitting moiety precursor is readily set free after oxidation of the leaving group precursor. Upon the action of peroxide or a reactive oxygen species like the oxygen radical anion the precursor of the light emitting moiety according to the mechanism illustrated in FIG. 2 most likely forms a dioxetane intermediate which is decarboxylated to generate an electronically excited emitter. The transition to the ground state of this emitter ensues by emission of a photon (=chemiluminescence). The energy (light) which is thereby emitted is measured according to standard procedures and with routine equipment.

As indicated, H₂O₂ or a reactive oxygen species like the oxygen radical anion has to be present to form the intermediate dioxetanone. H₂O₂ can be added directly or generated indirectly e.g. by enzymatic reaction (glucose oxidase/glucose). Reactive oxygen species are generated during the chemiluminescent reaction from oxygen or H₂O₂. Alternatively, a reactive oxygen species can be generated intentionally e.g. by the oxygen initiated C—C coupling (indoxyl-phosphate, U.S. Pat. No. 5,589,328).

The mentioned oxidation steps, e.g., catalyzed by enzymes like peroxidase can also be accelerated by the use of mediators or enhancers.

Mediators are redox-active compounds which facilitate the oxidation of a compound by accelerating electron transfer processes. The mediator is oxidized by the oxidant and oxidizes then the compounds according to the invention, whereby the mediator is reduced again. Typical mediators are hexocyanoferrate (II) and metal complexes like ferrocene. Other enhancers which are used in chemiluminescense reactions include chemicals like iodo-phenol or phenyl boronic acid.

The oxidation preferably is performed in the presence of an appropriate detergent, which creates a hydrophobic microenvironment around the light emitting heterocyclic ketone. This results in an increase of the chemiluminescence quantum yield since quenching due to interaction with water molecules is reduced. Additionally an appropriate fluorophor, like fluorescein can be attached covalent to the detergent or alternatively a fluorophor can be added to the reaction mixture in order to get an energy transfer from the excited heterocyclic ketone to this fluorophor.

It represents an additional attractive feature of the compounds described in the present invention that quite different reaction kinetics can be generated and compounds selected as required. This becomes evident by comparing the emission kinetics as shown in FIGS. 3 and 4. A glow type reaction kinetics (slow but long lasting reaction) is very preferred in applications like the blotting techniques. Use of a glow type compounds according to the present invention for staining in conjunction with a blotting technique also represents a preferred embodiment.

Most preferred the flash type compounds (fast light emission in form of a high intensity peak) are used in liquid phase immunoassays.

The following examples, references, and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 Luminescence reaction based on luciferin

The following abbreviations are used: ATP=adenosine tri-phosphate; AMP=adenosine mono-phosphate; HS-CoA=co-enzym A.

FIG. 2 Proposed mechanism of luminescence reaction for luciferin hydrazides

The terminus “nucl.” Shall indicate an intermediate nucleophilic substitution.

FIG. 3 Synthesis pathways for Dimethyl-D-luciferin hydrazide and Dimethyl-D-luciferin phenyl hydrazide

The following abbreviations are used: TBDMS=tert.-butyl di-methylsilyl chloride; (COCl)₂=oxalyl chloride; DMF=di-methyl formamide; TFA=tri-fluoro acetic acid; TBAF=tetrabutyl ammonium fluoride; Z (1-5) represents one or several substituents at the phenolic ring.

FIG. 4 Luminescence reaction of Dimethyl-D-luciferin hydrazide

RLU=relative light absorbance unit

FIG. 5 Luminescence reaction of Dimethyl-D-luciferin phenyl hydrazide

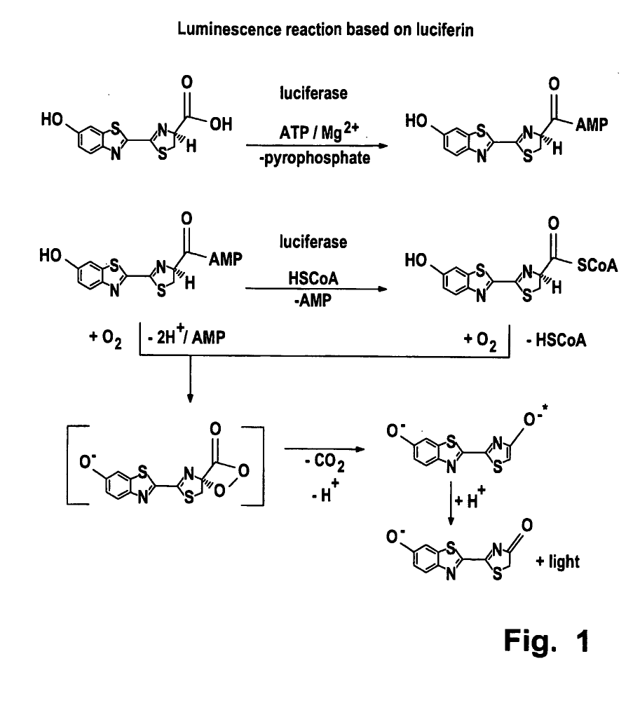

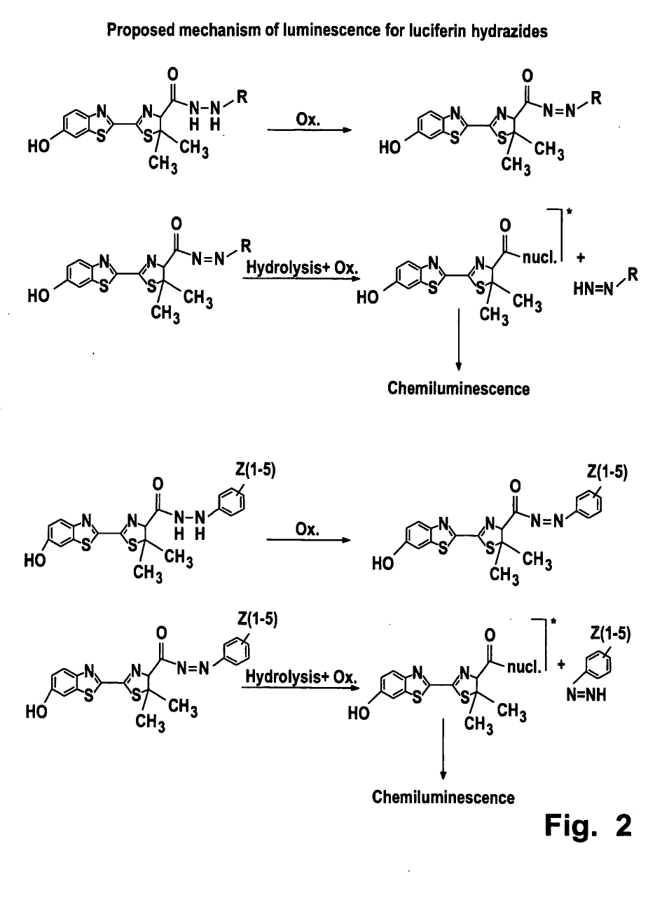

RLU=relative light absorbance unit

### EXAMPLES

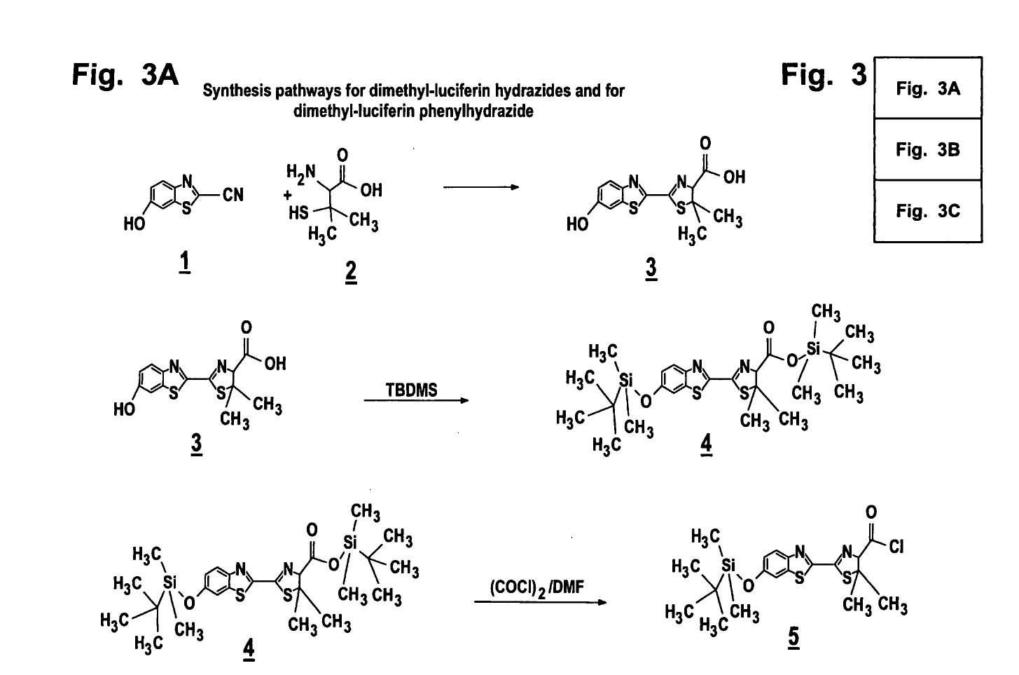

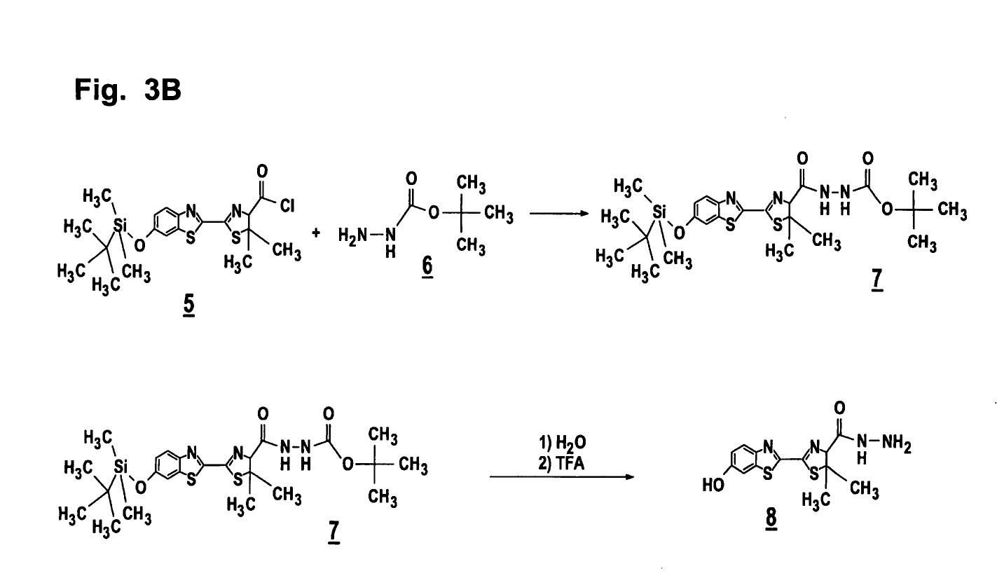

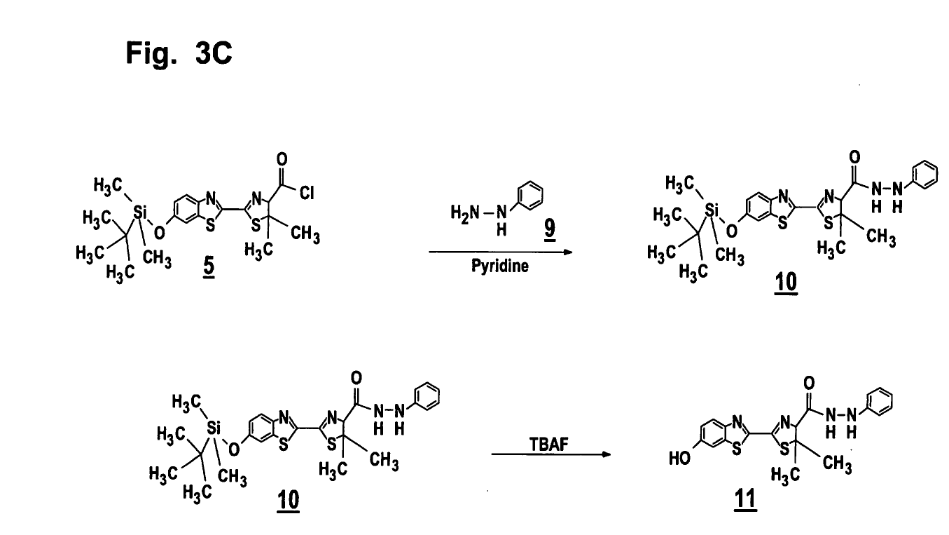

(The synthesis pathways for Examples 1 and 2 are also given FIG. 3. The underlined numbers in examples 1 and 2 correspond to chemical structures depicted there.)

### Example 1

### Synthesis of Dimethyl-D-luciferin hydrazide[4,5-Dihydro-2-(6-hydroxybenzo-thiazol-2-yl)-5,5-dimethylthiazole-4-yl-carboxylic acid hydrazide]

a) Preparation of dimethylluciferin, (4,5-dihydro-2-(6-hydroxybenzothiazole-2-yl)-5,5-dimethylthiazole-4-yl carboxylic acid) (3=structure 3 in FIG. 3)

704 mg (4 mmol) 2-cyano-6-hydroxybenzothiazole 1(prepared according to EP 0024525), 596 mg (4 mmol) D-penicillamine 2(Aldrich, no. P40-3) and 276 mg (2 mmol) potassium carbonate are dissolved in 6 ml methanol and 3,2 ml distilled water under argon atmosphere. While stirring the mixture is refluxed for 3 h to obtain a dear yellow liquid.

By using a rotary evaporator the solvents are removed at reduced pressure (water bath 40° C.). The remaining yellow brownish suspension is taken up in 100 ml distilled water and pH is adjusted to 2 with conc. hydrochloric acid. The desired product precipitates and is filtered off using a sintered glass funnel. The residue is rinsed out into a flask with a small volume of methanol. Subsequently the methanol is removed by using a rotary evaporator under reduced pressure (water bath 40° C.) to obtain a yellow solid.

Analysis, like for many of the following compounds, is performed by thin layer chromatography=TLC.

TLC (Kieselgel 60 F254, methanol/chloroform 1/1): Rf=0.81

b) Preparation of 2-(6-tert.-butyldimethylsilyloxybenzothiazole-2-yl)-4,5-dihydro-5,5-dimethylthiazole-4-yl carboxylic acid chloride (structure 5 in FIG. 3)

385 mg (1.25 mmol)dimethyl-D-luciferin 3 are dissolved in 50 ml dry tetrahydrofurane under argon atmosphere. 415 mg (2.75 mmol) tert.-butyldimethylsilyl chloride (Aldrich, no. 19,050-0) and subsequently 0,506 ml (5,0 mmol)triethyl amine are added at ambient temperature. After a few minutes a white precipitate of ammonium chloride is forming. The solution is stirred under argon atmosphere overnight, then the precipitate is filtered off and the solvent removed on a rotavapor (water bath 40° C.) to obtain 2-(6-tert.-butyldimethylsilyloxybenzothiazole-2-yl)-5,5-dimethyl-4,5-dihydrothiazole-4-yl carboxylic acid tert.-butyl dimethylsilyl ester; (structure 4 in FIG. 3) as a yellow slightly brownish oil.

2-(6-tert.-butydimethylsilyloxybenzothiazole-2-yl)-4,5-dihydro-5,5-dimethyl-4-thiazole carboxylic acid is dissolved under argon atmosphere in 8 ml dry methylene chloride, the clear solution is cooled to −15° C. and a mixture of 0.162 ml (1,875 mmol)oxalyl chloride and 500 μl of freshly distilled dimethyl formamide are added dropwise to the reaction under vigorous stirring. Slight gas emission can be observed during this step. The reaction mixture is stirred for additional 30 minutes at −15° C. and subsequently diluted with freshly destilled methylene chloride to a final volume of 30 ml.

The resulting dear brownish red solution of 2-(6-tert.-butyldimethylsilyloxybenzothiazole-2-yl)-4,5-dihydro-5,5-dimethyl-4-thiazole carboxylic acid chloride 5 is directly used in the next step without further purification.

c) Preparation of N-BOC-Dimethyl-D-luciferin hydrazide[4,5-Dihydro-2-(6-hydroxybenzo-thiazole-2-yl)-5,5-dimethylthiazole-4-yl-carboxylic acid 2-(1,1-dimethylethoxycarbonyl) hydrazide 7

A solution of 1.25 mmol 2-(6-tert.-butyldimethylsilyloxybenzothiazole-2-yl)-4,5-dihydro-5,5-dimethyl-4-thiazole carboxylic acid chloride 5 obtained from the preceding experiment is allowed to come to room temperature. 333 mg (2.5 mmol) tert.-butyl carbazate 6 (Aldrich, no. B-9,100-5) in 5 ml freshly destilled tetrahydrofurane and 250 μmol (2.5 mmol)triethyl amine are added. Precipitation of ammonium salt will be observed and the suspension is heated to 60° C. on an oil bath for 4 h. The mixture is cooled to room temperature, the precipitate removed by filtration and the solution evaporated. About 1 g of a red oil is obtained. It is applied to a preparative reversed phase HPLC system (Vydac C-18 column, 300 Å, 15-20 μm, 50×250 mm) in 200 mg portions. The product is eluted with an acetonitrile/distilled water gradient (0-100% acetonitrile; 0.1% trifluoroacetic acid). Most of the silyl protecting group at pos. 6 is removed during this work-up procedure and the desilylated product is obtained together with little silylated product.

The appropriate fractions are collected and pooled. Finally the solvent is removed by lyophilisation to obtain 60 mg slightly green product 7.

TLC (Kieselgel 60 F₂₅₄, ethyl acetate/methanol 1/1): R_{f}=0.89

d) Preparation of Dimethyl-D-luciferin hydrazide[4,5-Dihydro-2-(6-hydroxybenzo-thiazole-2-yl)-5,5-dimethylthiazole-4-yl-carboxylic acid hydrazide 8

54 mg (100 μmol) BOC protected hydrazide 7 is dissolved in 2 ml dry tetrahydrofurane and cooled to 0° C. on an ice bath. 2 ml trifluoro acetic acid are added and then the ice bat is removed. After stirring the clear yellow solution for 1 h, the solvent is evaporated and the residue lyophilized from dioxane. The pure product 8 is obtained as off-white solid (yield: 44 mg).

TLC (Kieselgel 60 F₂₅₄, ethyl acetate/methanol 1/1): R_{f=}0.48

Molecular weight has been confirmed by electrospray ionization mass spectroscopy=ESI-MS. ESI-MS:M+=322

### EXAMPLE 2

### Synthesis of Dimethyl-D-luciferin phenylhydrazide[4,5-Dihydro-2-(6-hydroxybenzo-thiazole-2-yl-5,5-dimethylthiazole-4-yl-carboxylic acid 2-phenylhydrazide

a) Preparation 4,5-Dihydro-2-(6-tert.-butyldimethylsilyloxybenzothiazole-2-yl)-5,5-dimethylthiazole-4-yl-carboxylic acid-2-phenylhydrazide 10

A solution of 1.25 mmol 2-(6-tert.-butyldimethylsilyloxybenzothiazole-2-yl)-4,5-dihydro-5,5-dimethyl-4-thiazole carboxylic acid chloride 5 is triturated with a mixture of 5 ml pyridine, 2 ml dimethylformamide and 395 μl (4 mmol)phenyl hydrazine 9 (Merck eurolab, no. 107251), all components freshly destilled. An instant formation of precipitation under change of color to orange is observed. The mixture is allowed to come to room temperature and stirred for 48 h. The solid is removed by filtration and the clear solution evaporated (oil pump vacuum, water bath 40° C.). The product is separated from the remaining deep orange oil by preparative reversed phase HPLC (Waters Delta Pak C-18 column, 100 Å, 15 μm, 50×300 mm). The product is eluted with an acetonitrile/distilled water gradient (0-70% acetonitrile; 0.1% trifluoro acetic acid). The appropriate fractions are collected and pooled. Finally the solvent is removed by lyophilisation to obtain 121 mg of orange product 10.

TLC (Kieselgel 60 F₂₅₄, petrol ether/ethyl acetate 1/1): R_{f}=0.87

b) Preparation of Dimethyl-D-luciferin phenylhydrazide[4,5-Dihydro-2-(6-hydroxybenzo-thiazole-2-yl)-5,5-dimethylthiazole-4-yl-carboxylic acid 2-phenylhydrazide 11

102 mg (200 μmol) of silylated hydrazide 10 are dissolved in 15 ml freshly destined tetrahydrofurane under argon and 105 mg (400 μmol)tetrabutylammonium fluoride monohydrate (Aldrich, no. 24,151-2) are added. The reaction vessel is sealed and the solution stirred at room temperature for 1 h. 20 ml of dichloromethane are added, then the mixture is transferred to a separator funnel and washed with 2×10 ml 5% ammonium chloride solution and subsequently 2×10 ml saturated sodium bicarbonate. The solvent is evaporated and the crude product is purified by preparative HPLC (Waters Delta Pak C-18 column, 100 Å, 15 μm, 50×300 mm). The product is eluted with an acetonitrile/distilled water gradient (0-70% acetonitrile; 0.1% trifluoro acetic acid). The appropriate fractions are pooled and the product 11 is obtained as slightly green solid after lyophilsation (yield: 14 mg).

TLC (Kieselgel 60 F₂₅₄, petrol ether/ethyl acetate 1/1): R_{f}=0.75

Molecular weight has been confirmed by electrospray ionization mass spectroscopy=ESI-MS. ESI-MS: M+=398.

### Example 3

### Chemiluminescence

Measurements were performed on a Berthold Lumat LB953. To produce chemiluminescence two triggers have been used. Trigger 1 brings about the oxidation of the leaving group precursor, trigger 2 promotes chemiluminescence.

Trigger 1:300 μl, 0.5% H2O2, 0.1M HNO3

Trigger 2:300 μl, 0.25M NaOH

The luciferin carbonyl hydrazides according to Examples 1 and 2, respectively, were diluted to 1×10⁻⁸ Mol/l in PBS-buffer containing 0.1% Thesit. 100μl sample was dispensed into a 5 ml-Sarstedt tube and set into the instrument. Trigger 1 was added in position-1, trigger 2 in the measuring position of the instrument. Measurement was performed for 10 sec.

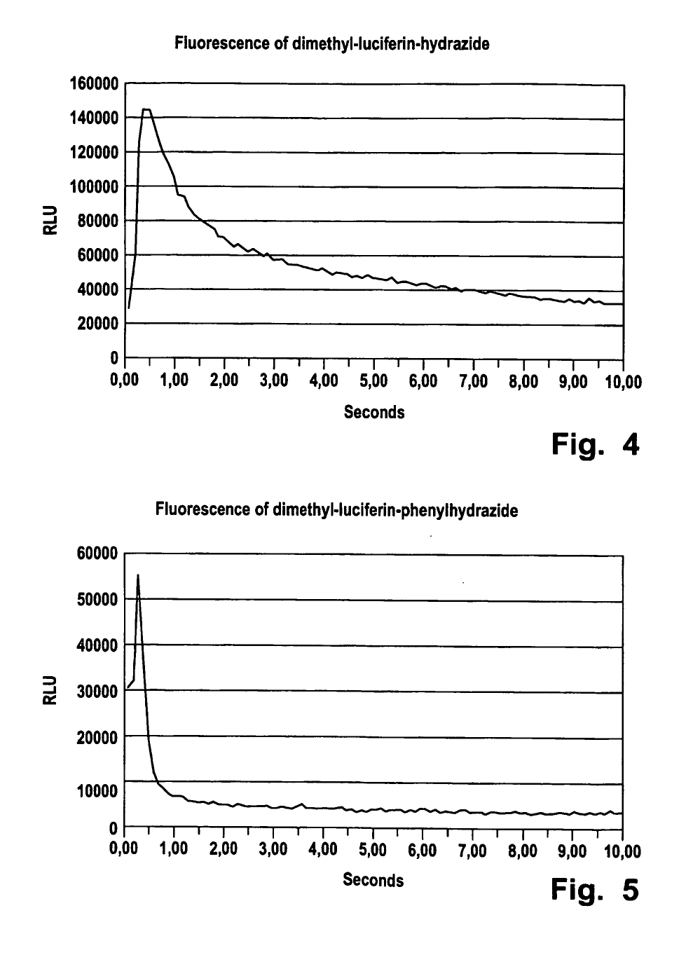

The kinetics of light emission for the compounds synthesized in Examples 1 and 2 are given in FIGS. 4 and 5, respectively. As can be seen, the phenyl substitution leads to a sharper peak of fluorescence.

### LIST OF REFERENCES

Aslam, M. and Dent, A., The preparation of protein-protein conjugates in “Bioconjugation” (1998) 216-363, Eds. M. Aslam and A. Dent, McMillan, London

Huenig, S., Pure & Appl. Chem. 62 (1990) 396-406

Kessler, C., in “Non-radioactive labeling and detection of biomolecules” (1992), Springer Verlag, Heidelberg

Mayer, A. and Neuenhofer, S., Luminescent labels—more than just an alternative to radioisotopes? in “Angewandte Chemie: International Edition in English” (1994) 1044-1072, Eds. E. P. Goelitz, VCH Verlagsgesellschaft mbH, Weinheim

Tijssen, in “Methods in Enzymology”, Eds. S. P. Colowick, N. O. Caplan and S. P., Academic Press

Tijssen, in “Practice and theory of enzyme immunoassays” (1990), Elsevier, Amsterdam

U.S. Pat. No. 5,589,328

1-10. (cancelled)

11. A chemical compound comprising a luciferin class dye [or an analogue thereto] and a substituted hydrazide group, wherein a nitrogen atom of the hydrazide group is bound to a carbonyl or thionyl group of the dye [or to a group chemically equivalent to the carbonyl group] and wherein the hydrazide is substituted with a phenyl group comprising the substituents Z1, Z2, Z3, Z4, and Z5, each independently selected from the group consisting of H, Y, alky, alkyl-Y, aryl, aryl-Y, alky-aryl, alkyl-aryl-Y, hetreoaryl, heteroaryl-Y, OH, NH₂, O-alkyl, NH-alkyl, N(alkyl)₂, O-aryl, and halogen, [wherein two or more of the groups Z1-Z5 comprise a carbocyclic or heterocyclic ring system] and wherein Y is a coupling group or a label and is only present once where Y is a coupling group.

12. The compound of claim 11 wherein the dye is a dimethyl luciferin dye.

13. The compound of claim 11 wherein the nitrogen atom is bound to a carbonyl group of the dye.

14. The compound of claim 11 wherein the hydrazide is substituted with a group selected from the group consisting of alkyl, alkyl-Y, aryl, aryl-Y, alkyl-aryl, alkyl-aryl-Y, heteroaryl, and heteroaryl-Y, wherein Y is a coupling group or a label.

15. The compound of claim 14 wherein the aryl group is a C₆, C₁₀ or C₁₄ group.

16. A conjugate comprising the compound of claim 11 and a biomolecule.

17. A method for detection of an analyte in a sample via specific binding assay comprising
(a) reacting the sample with a conjugate of a biomolecule and a compound comprising a light emitting moiety precursor and a leaving group precursor linked together by a carbonyl-hydrazide bond, wherein the biomolecule binds specifically with the analyte,
(b) oxidizing the leaving group precursor bound to the analyte via the biomolecule, thereby setting free the signal emitting group and causing light to be emitted, and
(c) measuring the light emitted in step (b) as a measure of the analyte in the sample.

18. A method for luminescence measurement comprising
(a) providing a compound comprising a light emitting moiety precursor and a leaving group precursor linked together by a carbonyl-hydrazide bond,
(b) oxidizing the leaving group precursor, thereby setting free the signal emitting group and causing light to be emitted, and
(c) measuring the light emitted in step (b).
